(19) United States
(12) Patent Application Publication
(10) Pub. No.: US 2023/0351585 A9
(43) Pub. Date: Nov. 2, 2023

(54) ARTIFICIAL INTELLIGENCE ENABLED, PORTABLE, PATHOLOGY MICROSCOPE

(71) Applicant: Board of Regents, The University of Texas System, Austin, TX (US)

(72) Inventor: Scott Terry Moen, Galveston, TX (US)

(21) Appl. No.: 17/964,125

(22) Filed: Oct. 12, 2022

Related U.S. Application Data
(60) Provisional application No. 63/254,703, filed on Oct. 12, 2021.

(51) Int. Cl. G06T 7/00; G16H 50/30; G16H 50/20; H04N 5/225; G06T 7/11; G02B 21/36; G02B 21/00; G02B 21/06

(52) U.S. Cl. CPC G06T 7/0012; G16H 50/30; G16H 50/20; H04N 5/2256; G06T 7/11; G02B 21/367; G02B 21/0008; G02B 21/06; G06T 2207/20081; G06T 2207/10056; G06T 2207/30024; G06T 2207/20212; G06T 2207/20084

(57) ABSTRACT

A portable microscope includes an enclosure having an opening configured to receive a slide, a slide holder disposed within the enclosure and operably positioned with respect to the opening to receive the slide, a lens system disposed within the enclosure above the slide holder, a light source disposed within the enclosure below the slide holder, a camera disposed within the enclosure and optically aligned with the lens system, a processor disposed within the enclosure and communicably coupled to the camera, a display screen affixed to the enclosure and visible from an exterior of the enclosure, wherein the display screen is communicably coupled to the processor. The processor is configured to obtain an image of a specimen disposed on the slide, analyze the specimen using artificial intelligence, and display the image of the specimen and a result of the analysis on the display screen.

### CROSS-REFERENCE TO RELATED APPLICATIONS

This application claims priority to U.S. Provisional Application Ser. No. 63/254,703, filed Oct. 12, 2021 entitled “Artificial Intelligence Enabled, Portable, Pathology Microscope”, which is hereby incorporated by reference in its entirety.

### TECHNICAL FIELD OF THE INVENTION

The present invention relates generally to the field of microscopes and, more particularly, to an artificial intelligence enabled, portable, pathology microscope.

### INCORPORATION-BY-REFERENCE OF MATERIALS FILED ON COMPACT DISC

None.

### STATEMENT OF FEDERALLY FUNDED RESEARCH

None.

### BACKGROUND OF THE INVENTION

None.

### SUMMARY OF THE INVENTION

In one embodiment, a microscope includes an enclosure having an opening configured to receive a slide, a slide holder disposed within the enclosure and operably positioned with respect to the opening to receive the slide, a lens system disposed within the enclosure above the slide holder, a light source disposed within the enclosure below the slide holder, a camera disposed within the enclosure and optically aligned with the lens system, a processor disposed within the enclosure and communicably coupled to the camera, and a display screen affixed to the enclosure and visible from an exterior of the enclosure, wherein the display screen is communicably coupled to the processor. The processor is configured to obtain an image of a specimen disposed on the slide, analyze the specimen using artificial intelligence, and display the image of the specimen and a result of the analysis on the display screen. The microscope is portable.

In one aspect, the light source comprises an addressable ring-shaped LED-based light where intensity, color and pattern are controlled by the processor for sample illumination and excitation. In another aspect, the microscope further comprises one or more input/output connectors accessible from the exterior of the enclosure and communicably coupled to the processor. In another aspect, the microscope further comprises a power source disposed within the enclosure. In another aspect, the display screen comprises a touch screen display. In another aspect, the microscope further comprises a memory disposed within the enclosure and communicably coupled to the processor. In another aspect, the microscope further comprises an artificial intelligence processor communicably coupled to the processor. In another aspect, the artificial intelligence is trained to automatically prepare the image for subsequent analysis. In another aspect, the artificial intelligence is trained to perform an edge analysis of the specimen. In another aspect, a non-subject matter expert assesses the sample based on the analysis. In another aspect, the analysis comprises determining whether the image of the sample is potentially positive for a given condition and flagging the sample for further review by a subject-matter expert (SME). In another aspect, the potentially positive image is decreased in size. In another aspect, all the potentially positive images are transmitted to a device in a batch via a wired or wireless connection coupled to the processor. In another aspect, the processor prompts a user on how to insert the slide properly into the slide holder. In another aspect, the image comprises a series of images that are stitched together using the processor. In another aspect, the analysis comprises preselecting cellular architecture within the image by machine learning segmentation. In another aspect, the analysis comprises normalizing a stitched image brightness, intensity, or color of the image. In another aspect, the analysis comprises binning of image values to quantitate or qualify on a range of values, rather than discrete values. In another aspect, the artificial intelligence adjusts the image. In another aspect, the artificial intelligence comprises one or more qualitative or quantitative machine learning edge models. In another aspect, the artificial intelligence comprises a NIH Image J plugin that quantifies bio-marker signal densities and consequently cancer risk. In another aspect, the artificial intelligence comprises a convolutional neural net (CNN) partially trained on bio-marker images to analyze for cancer risk. In another aspect, the analysis comprises one or more of: a differential White Blood Cell (WBC) count on a patient's blood smear or urine sample or bone marrow smear using Wright Stain; a qualification of a Gram-Stained blood smear from a bacteremic patient; a qualification of a Silver-Stained blood smear from a patient suspected of having a spirochete infection; a qualification and quantification of a periodic acid-Schiff staining procedure on a liver sample for a patient suspected of having glycogen storage disease; a quantification and patterning of Prussian Blue on a liver biopsy slide of a patient suspected of having hemochromatosis; a qualification of a Gomori Trichrome Stain for a patient suspected of having liver cirrhosis; a qualification of a Hematoxylin and Eosin (H&E) Stain on a polyp biopsy slide for a patient suspected of having cancer; a quantification of a co-localization of multiple colors, as is the case for FRET; a quantitation of specific cell types; a quantification of cell morphologies; or an assessment of tissue health.

In another embodiment, a method includes: providing a portable microscope comprising an enclosure having an opening configured to receive a slide, a slide holder disposed within the enclosure and operably positioned with respect to the opening to receive the slide, a lens system disposed within the enclosure above the slide holder, a light source disposed within the enclosure below the slide holder, a camera disposed within the enclosure and optically aligned with the lens system, a processor disposed within the enclosure and communicably coupled to the camera, and a display screen affixed to the enclosure and visible from an exterior of the enclosure, wherein the display screen is communicably coupled to the processor; placing the slide into the slide holder and positioning a sample on the slide within an optical path of the lens system and the camera; capturing an image of the sample using the camera; analyzing the sample using an artificial intelligence with the processor; and displaying the image of the specimen and a result of the analysis on the display screen.

In one aspect, the method further comprising preparing the slide with a hematoxylin and eosin (H&E) staining procedure using CLICK-S antibody. In another aspect, the light source comprises an addressable ring-shaped LED-based light where intensity, color and pattern are controlled by the processor for sample illumination and excitation. In another aspect, one or more input/output connectors are accessible from the exterior of the enclosure and communicably coupled to the processor. In another aspect, a power source disposed within the enclosure. In another aspect, the display screen comprises a touch screen display. In another aspect, a memory is disposed within the enclosure and communicably coupled to the processor. In another aspect, an artificial intelligence processor is communicably coupled to the processor. In another aspect, the artificial intelligence is trained to automatically prepare the image for subsequent analysis. In another aspect, the artificial intelligence is trained to perform an edge analysis of the specimen.

In another aspect, the method further comprises assessing the sample based on the analysis by a non-subject matter expert. In another aspect, analyzing the sample using the artificial intelligence comprises determining whether the image of the sample is potentially positive for a given condition and flagging the sample for further review by a subject-matter expert (SME). In another aspect, the method further comprised decreasing the potentially positive image in size. In another aspect, the method further comprises transmitting all the potentially positive images to a device in a batch via a wired or wireless connection coupled to the processor. In another aspect, the method further comprised prompting a user on how to insert the slide properly into the slide holder. In another aspect, the method further comprises stitching together a series of images together into the image using the processor. In another aspect, analyzing the sample using the artificial intelligence comprises preselecting cellular architecture within the image by machine learning segmentation. In another aspect, analyzing the sample using the artificial intelligence comprises normalizing a stitched image brightness, intensity, or color of the image. In another aspect, analyzing the sample using the artificial intelligence comprises binning of image values to quantitate or qualify on a range of values, rather than discrete values. In another aspect, analyzing the sample using the artificial intelligence comprises adjusting the image. In another aspect, the artificial intelligence comprises one or more qualitative or quantitative machine learning edge models. In another aspect, the artificial intelligence comprises a NIH Image J plugin that quantifies bio-marker signal densities and consequently cancer risk. In another aspect, the artificial intelligence comprises a convolutional neural net (CNN) partially trained on bio-marker images to analyze for cancer risk. In another aspect, analyzing the sample using the artificial intelligence comprises one or more of: performing a differential White Blood Cell (WBC) count on a patient's blood smear or urine sample or bone marrow smear using Wright Stain using the artificial intelligence; performing a qualification of a Gram-Stained blood smear from a bacteremic patient using the artificial intelligence; performing a qualification of a Silver-Stained blood smear from a patient suspected of having a spirochete infection using the artificial intelligence; performing a qualification and quantification of a periodic acid-Schiff staining procedure on a liver sample for a patient suspected of having glycogen storage disease using the artificial intelligence; performing a quantification and patterning of Prussian Blue on a liver biopsy slide of a patient suspected of having hemochromatosis using the artificial intelligence; performing a qualification of a Gomori Trichrome Stain for a patient suspected of having liver cirrhosis using the artificial intelligence; performing a qualification of a Hematoxylin and Eosin (H&E) Stain on a polyp biopsy slide for a patient suspected of having cancer using the artificial intelligence; performing a quantification of a co-localization of multiple colors, as is the case for FRET using the artificial intelligence; performing a quantitation of specific cell types using the artificial intelligence; performing a quantification of cell morphologies using the artificial intelligence; or performing an assessment of tissue health using the artificial intelligence.

The present invention is described in detail below with reference to the accompanying drawings.

### BRIEF DESCRIPTION OF THE DRAWINGS

The above and further advantages of the invention may be better understood by referring to the following description in conjunction with the accompanying drawings, in which:

FIG. 1 is a block diagram of a microscope according to an embodiment of the invention;

FIG. 2 is a flow chart of a method in according to an embodiment of the invention;

FIG. 3 is an image of an interior of a microscope in accordance with one embodiment of the invention;

FIG. 4 is an image of a touchscreen display of a microscope in accordance with one embodiment of the invention;

FIGS. 5A and 5B are images illustrating the microscope prompting a user to insert slide into the microscope in accordance with one embodiment of the invention; and

FIG. 6 is a block diagram of a method in accordance with one embodiment of the invention.

### DETAILED DESCRIPTION OF THE INVENTION

The current invention now will be described more fully hereinafter with reference to the accompanying drawings, which illustrate embodiments of the invention. This invention may, however, be embodied in many different forms and should not be construed as limited to the illustrated embodiments set forth herein. Rather, these embodiments are provided so that this disclosure will be thorough and complete, and will fully convey the scope of the invention to those skilled in the art.

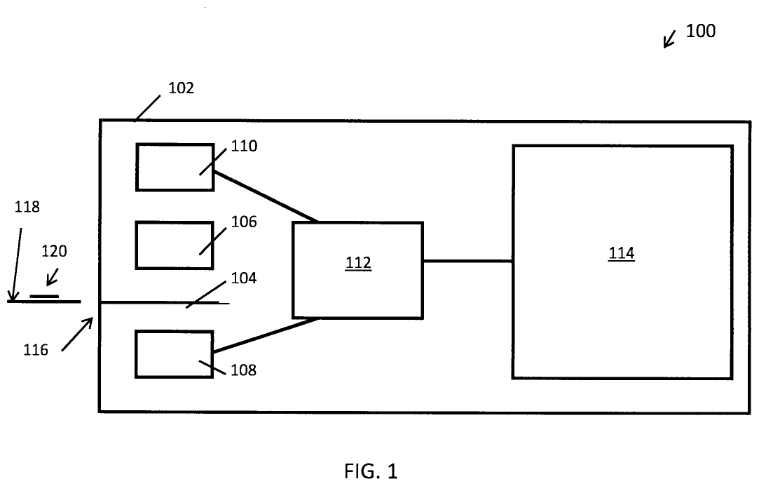

FIG. 1 is a block diagram of a microscope 100 in accordance with one embodiment of the present invention. The microscope 100 includes an enclosure 102, a slide holder 104, a lens system 106, a light source 108, a camera 110, a processor 112, and a display screen 114. The enclosure 102 has an opening 116 configured to receive a slide 118. The slide holder 104 is disposed within the enclosure 102 and is operably positioned with respect to the opening 116 to receive the slide 118. The lens system 106 is disposed within the enclosure 102 above the slide holder 104. The light source 108 is disposed within the enclosure 102 below the slide holder 104. The camera 110 is disposed within the enclosure 102 and is optically aligned with the lens system 106. The processor 112 is disposed within the enclosure 102 and is communicably coupled to the camera 110 and the light source 108. The display screen 114 is affixed to the enclosure 102 and is visible from an exterior of the enclosure 102. The display screen 114 is also communicably coupled to the processor 112. The processor 112 is configured to obtain an image of a specimen 120 disposed on the slide 118, analyze the specimen 120 using artificial intelligence, and display the image of the specimen and a result of the analysis on the display screen 114. The microscope 100 is portable.

In one aspect, the light source 108 comprises an addressable ring-shaped LED-based light where intensity, color and pattern are controlled by the processor 112 for sample illumination and excitation. In another aspect, the microscope 100 further comprises one or more input/output connectors accessible from the exterior of the enclosure 102 and communicably coupled to the processor 112. In another aspect, the microscope 100 further comprises a power source disposed within the enclosure 102. In another aspect, the display screen 114 comprises a touch screen display. In another aspect, the microscope 100 further comprises a memory disposed within the enclosure 102 and communicably coupled to the processor 112. In another aspect, the microscope 100 further comprises an artificial intelligence processor communicably coupled to the processor 112. In another aspect, the artificial intelligence is trained to automatically prepare the image for subsequent analysis. In another aspect, the artificial intelligence is trained to perform an edge analysis of the specimen. In another aspect, a non-subject matter expert assesses the sample based on the analysis. In another aspect, the analysis comprises determining whether the image of the sample specimen 120 is potentially positive for a given condition and flagging the sample for further review by a subject-matter expert (SME). In another aspect, the potentially positive image is decreased in size. In another aspect, all the potentially positive images are transmitted to a device in a batch via a wired or wireless connection coupled to the processor 112. In another aspect, the processor 112 prompts a user on how to insert the slide properly into the slide holder. In another aspect, the image comprises a series of images that are stitched together using the processor 112. In another aspect, the analysis comprises preselecting cellular architecture within the image by machine learning segmentation. In another aspect, the analysis comprises normalizing a stitched image brightness, intensity, or color of the image. In another aspect, the analysis comprises binning of image values to quantitate or qualify on a range of values, rather than discrete values. In another aspect, the artificial intelligence adjusts the image. In another aspect, the artificial intelligence comprises one or more qualitative or quantitative machine learning edge models. In another aspect, the artificial intelligence comprises a NIH Image J plugin that quantifies bio-marker signal densities and consequently cancer risk. In another aspect, the artificial intelligence comprises a convolutional neural net (CNN) partially trained on bio-marker images to analyze for cancer risk. In another aspect, the analysis comprises one or more of: a differential White Blood Cell (WBC) count on a patient's blood smear or urine sample or bone marrow smear using Wright Stain; a qualification of a Gram-Stained blood smear from a bacteremic patient; a qualification of a Silver-Stained blood smear from a patient suspected of having a spirochete infection; a qualification and quantification of a periodic acid-Schiff staining procedure on a liver sample for a patient suspected of having glycogen storage disease; a quantification and patterning of Prussian Blue on a liver biopsy slide of a patient suspected of having hemochromatosis; a qualification of a Gomori Trichrome Stain for a patient suspected of having liver cirrhosis; a qualification of a Hematoxylin and Eosin (H&E) Stain on a polyp biopsy slide for a patient suspected of having cancer; a quantification of a co-localization of multiple colors, as is the case for FRET; a quantitation of specific cell types; a quantification of cell morphologies; or an assessment of tissue health.

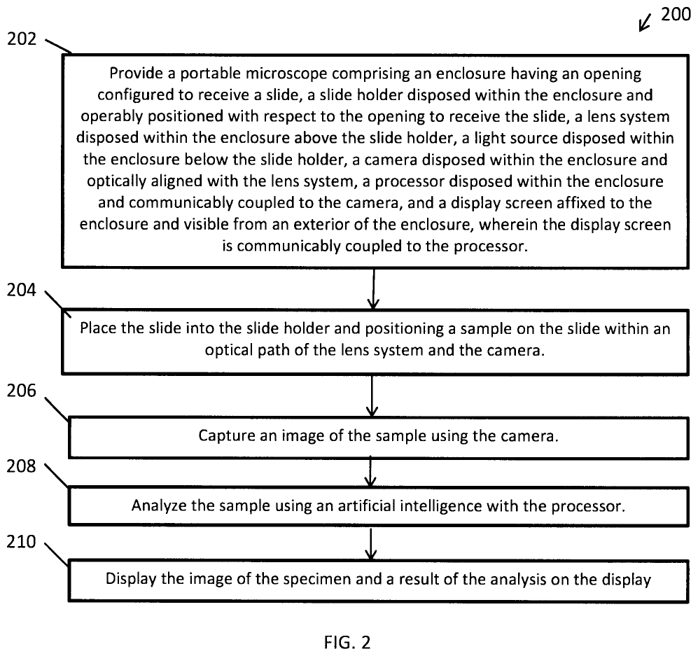

Now referring to FIG. 2, a method 200 in accordance with one embodiment of the present invention is shown. A portable microscope is provided in block 202. The portable microscope comprises an enclosure having an opening configured to receive a slide, a slide holder disposed within the enclosure and operably positioned with respect to the opening to receive the slide, a lens system disposed within the enclosure above the slide holder, a light source disposed within the enclosure below the slide holder, a camera disposed within the enclosure and optically aligned with the lens system, a processor disposed within the enclosure and communicably coupled to the camera, and a display screen affixed to the enclosure and visible from an exterior of the enclosure, wherein the display screen is communicably coupled to the processor. The slide is placed into the slide holder and a sample on the slide is positioned within an optical path of the lens system and the camera in block 204. An image of the sample is captured using the camera in block 206. The sample is analyzed using an artificial intelligence with the processor in block 208. The image of the specimen and a result of the analysis are displayed the display screen in block 210.

In one aspect, the method further comprising preparing the slide with a hematoxylin and eosin (H&E) staining procedure using CLICK-S antibody. In another aspect, the light source comprises an addressable ring-shaped LED-based light where intensity, color and pattern are controlled by the processor for sample illumination and excitation. In another aspect, one or more input/output connectors are accessible from the exterior of the enclosure and communicably coupled to the processor. In another aspect, a power source disposed within the enclosure. In another aspect, the display screen comprises a touch screen display. In another aspect, a memory is disposed within the enclosure and communicably coupled to the processor. In another aspect, an artificial intelligence processor is communicably coupled to the processor. In another aspect, the artificial intelligence is trained to automatically prepare the image for subsequent analysis. In another aspect, the artificial intelligence is trained to perform an edge analysis of the specimen. In another aspect, the method further comprises assessing the sample based on the analysis by a non-subject matter expert. In another aspect, analyzing the sample using the artificial intelligence comprises determining whether the image of the sample is potentially positive for a given condition and flagging the sample for further review by a subject-matter expert (SME). In another aspect, the method further comprised decreasing the potentially positive image in size. In another aspect, the method further comprises transmitting all the potentially positive images to a device in a batch via a wired or wireless connection coupled to the processor. In another aspect, the method further comprised prompting a user on how to insert the slide properly into the slide holder. In another aspect, the method further comprises stitching together a series of images together into the image using the processor. In another aspect, analyzing the sample using the artificial intelligence comprises preselecting cellular architecture within the image by machine learning segmentation. In another aspect, analyzing the sample using the artificial intelligence comprises normalizing a stitched image brightness, intensity, or color of the image. In another aspect, analyzing the sample using the artificial intelligence comprises binning of image values to quantitate or qualify on a range of values, rather than discrete values. In another aspect, analyzing the sample using the artificial intelligence comprises adjusting the image. In another aspect, the artificial intelligence comprises one or more qualitative or quantitative machine learning edge models. In another aspect, the artificial intelligence comprises a NIH Image J plugin that quantifies bio-marker signal densities and consequently cancer risk. In another aspect, the artificial intelligence comprises a convolutional neural net (CNN) partially trained on bio-marker images to analyze for cancer risk. In another aspect, analyzing the sample using the artificial intelligence comprises one or more of: performing a differential White Blood Cell (WBC) count on a patient's blood smear or urine sample or bone marrow smear using Wright Stain using the artificial intelligence; performing a qualification of a Gram-Stained blood smear from a bacteremic patient using the artificial intelligence; performing a qualification of a Silver-Stained blood smear from a patient suspected of having a spirochete infection using the artificial intelligence; performing a qualification and quantification of a periodic acid-Schiff staining procedure on a liver sample for a patient suspected of having glycogen storage disease using the artificial intelligence; performing a quantification and patterning of Prussian Blue on a liver biopsy slide of a patient suspected of having hemochromatosis using the artificial intelligence; performing a qualification of a Gomori Trichrome Stain for a patient suspected of having liver cirrhosis using the artificial intelligence; performing a qualification of a Hematoxylin and Eosin (H&E) Stain on a polyp biopsy slide for a patient suspected of having cancer using the artificial intelligence; performing a quantification of a co-localization of multiple colors, as is the case for FRET using the artificial intelligence; performing a quantitation of specific cell types using the artificial intelligence; performing a quantification of cell morphologies using the artificial intelligence; or performing an assessment of tissue health using the artificial intelligence.

Various non-limiting examples of the present invention will now be described with respect to both hardware and software.

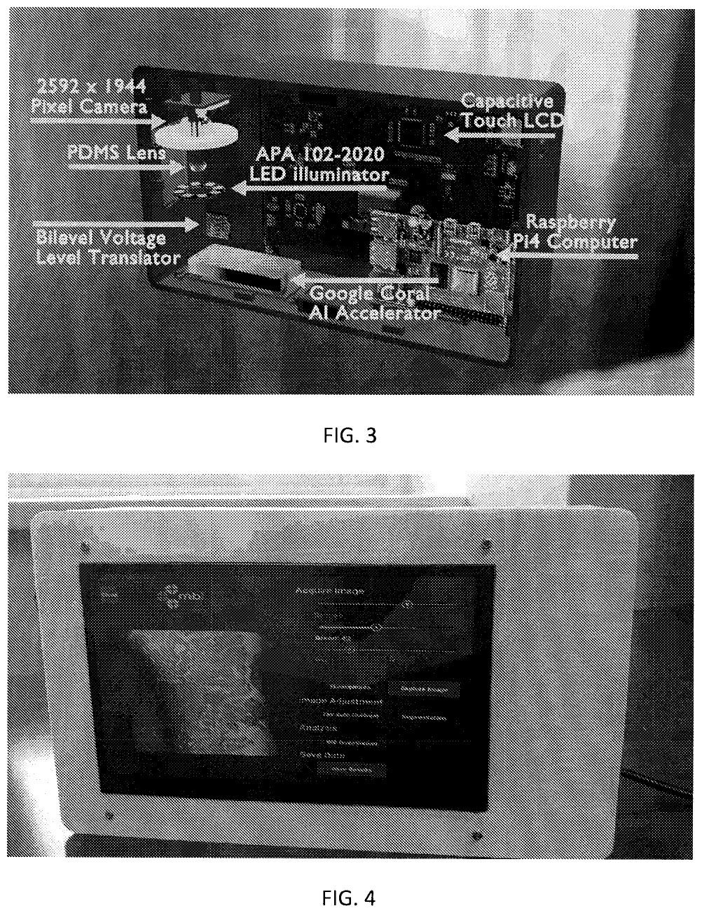

Referring now to FIG. 3, an image of an interior of a microscope in accordance with one embodiment of the invention is shown. The microscope includes a processor (e.g., Raspberry Pi 4, etc.), a lens system (e.g., a polydimethylsiloxane lens system, etc.), a artificial intelligence processor (e.g., a Google Coral ASIC, etc.), a camera, and a display screen (e.g., a capacitive touchscreen, etc.) in a battery-powered, relatively inexpensive, mobile Edge-TPU device. The significance of this is that this microscope can utilize AI without utilizing the Internet; it is able to perform its function for extended periods of time in resource-poor areas, while the components are inexpensive in comparison to current equivalents, as to be affordable to previously inaccessible markets.

The microscope can use AI to prepare the microscopy tissue images for subsequent analysis and/or it will utilize artificial intelligence/machine learning models trained on pathology slides to create models for edge inference on the Google Coral ASIC device. The software is designed to assist inexperienced users filter patient samples so that samples deemed an edge case (True Positive or mildly False Negative, as determined by logistic regression for the particular analysis) may be passed on to a more experienced professional for review.

Following the standard pathology workflow of pathology slide (e.g., paraffin embedding, sample slicing, and mounting of the slice on a microscope slide) or blood smear prep; the prepared slide will be used on the Artificial Intelligence (AI)-enabled, portable microscope. The system is designed to allow non-subject matter experts (SMEs) to assess pathology samples, where samples deemed positive for a given condition, by a trained machine learning or deep learning model, will be flagged for further review by an SME. In this manner, many pathology slides can be reviewed at remote locations, where the slides are generated and the potentially positive images can be sent in batch to the SME, when convenient. This system relieves the burden on the SME by not having to review as many samples while also increasing the availability of analysis to underserved areas.

The user of the device is assisted in two steps of the workflow: image preparation and sample analysis. During the image preparation phase the user is prompted on how to insert the slide properly for the microscope lens/camera system to acquire a series of images that an algorithm will stitch into one large image. This serves as the image to assessed. Next, the stitched image can be further prepared to assist in the eventual analysis. This preparation can involve the preselection of cellular architecture, by machine learning segmentation, to be used in the subsequent analysis; thereby masking the structures inappropriate to the desired analysis and removing a potential category of false positives. Another preparation method can be used to normalize the stitched image brightness, intensity, or color. Another preparation method can be the binning of image values to quantitate or qualify on a range of values, rather than discrete values.

Once the preparation phase has been completed, the image can be analyzed. Analysis can be a qualitative or quantitative machine learning edge models developed on a centralized hub, released initially with the microscope's computer operating system (OS) but updated periodically concurrently with the OS revisions. Another example application would be to perform a differential White Blood Cell (WBC) counts on a patient's blood smear (or urine sample or bone marrow smear) using Wright Stain. Irregularities in WBC count differentials can be a sign of an infection or autoimmune disease and can be used to determine the appropriate treatment (e.g., antibiotics or immune-suppressive drugs, respectively).

Another example application would the qualification of a Gram-Stained blood smear from a bacteremic patient. The Gram Stain would determine the presence of gram-positive or gram-negative bacteria, which would allow for the correct (effective) antibiotic to be prescribed.

Another example application would be the qualification of a Silver-Stained blood smear from a patient suspected of having a spirochete infection. These bacteria are notoriously difficult to stain with the Gram Stain but can be stained with Silver Stain. This diagnosis would then be used to prescribe the correct family of antibiotics.

Another example application would be the qualification and quantification of the periodic acid-Schiff staining procedure on a liver sample for a patient suspected of having glycogen storage disease. This diagnosis would lead to the proper management of the condition to include dietary restrictions.

Another example application would be the quantification and patterning of Prussian Blue on a liver biopsy slide of a patient suspected of having hemochromatosis. This diagnosis would lead to managed care targeting the removal of excess iron.

Another example application would be the qualification of Gomori Trichrome Stain for a patient suspected of having liver cirrhosis. This diagnosis could lead to the proper management of the condition such as reducing alcohol intake, changing the patient's diet, or treating for infection, depending on the cause.

Another example application would be the qualification of a Hematoxylin and Eosin (H&E) Stain on a polyp biopsy slide for a patient suspected of having cancer. This diagnosis could lead to proper management of the conditions such as referring the patient to an oncologist or scheduling the patient for a surgical intervention, depending on the local resources.

Another algorithm could be used to quantify the co-localization of multiple colors, as is the case for FRET. Another machine learning algorithm could be used to quantitate specific cell types. Another algorithm could be used to quantify cell morphologies. Another algorithm could be used to assess tissue health.

Once an image has been flagged for further review, the high resolution image that was used for analysis will be decreased in size for storage and subsequent batch transfer, when the microscope is either connected to a LAN network or connected to Wi-Fi, while the original image is stored locally until a storage threshold is reached and the user is prompted to delete the cache.

The hardware of the system is designed to be a portable and inexpensive platform that can screen pathology slides. The microscope hardware consists of off-the-shelf, easily replaceable, general-purpose camera that interacts with its single-board computer. This computer can be Linux, iOS, Microsoft, or Android-based. The computer should have edge-computing capabilities inherently or be accessorized with such capabilities (such as the Coral Google ASIC device). The computer should have the capabilities to store the images for batch transfer and the relative full-size images while also being battery powered, which makes it portable. The lens system is a PDMS-based or other inexpensive system with a short working distance to be used in a small portable form factor. Illumination is provided by an inexpensive, addressable ring-shaped LED-based light where intensity, color and pattern can be controlled for sample illumination and excitation. The screen should be capacitive touch, so that gloved fingers can interact with it.

FIG. 4 is an image of a touchscreen display of a microscope in accordance with one embodiment of the invention. The touchscreen display displays an image of the sample, various color touch slide bar controls, fluorophores touch control, capture image touch control, yen auto contrast touch control, segmentation touch control, IHC quantification touch control, and save results touch control.

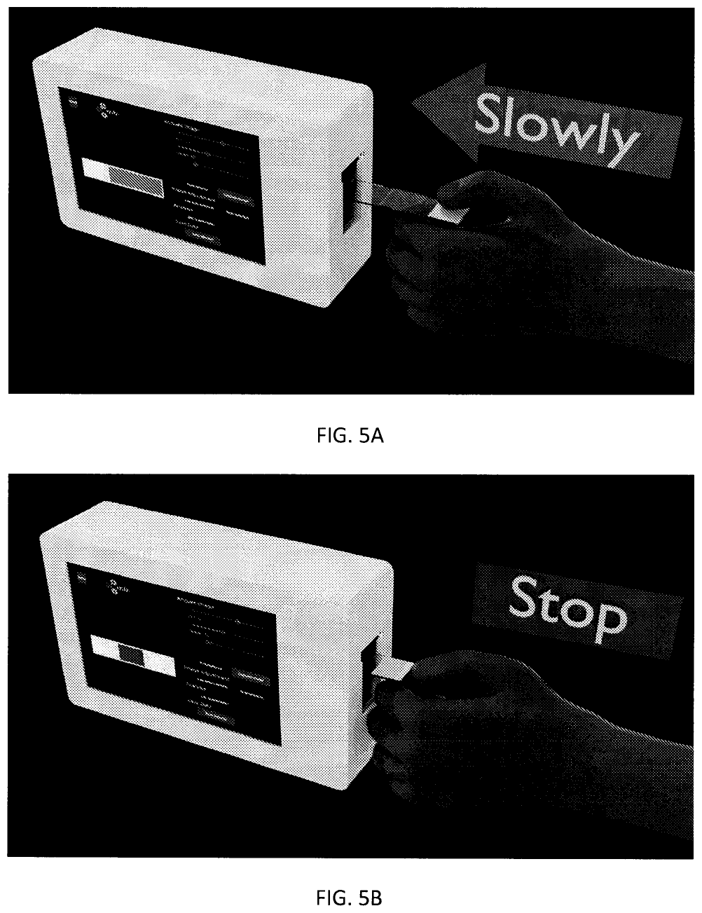

FIGS. 5A and 5B are images illustrating the microscope prompting a user to insert slide into the microscope in accordance with one embodiment of the invention.

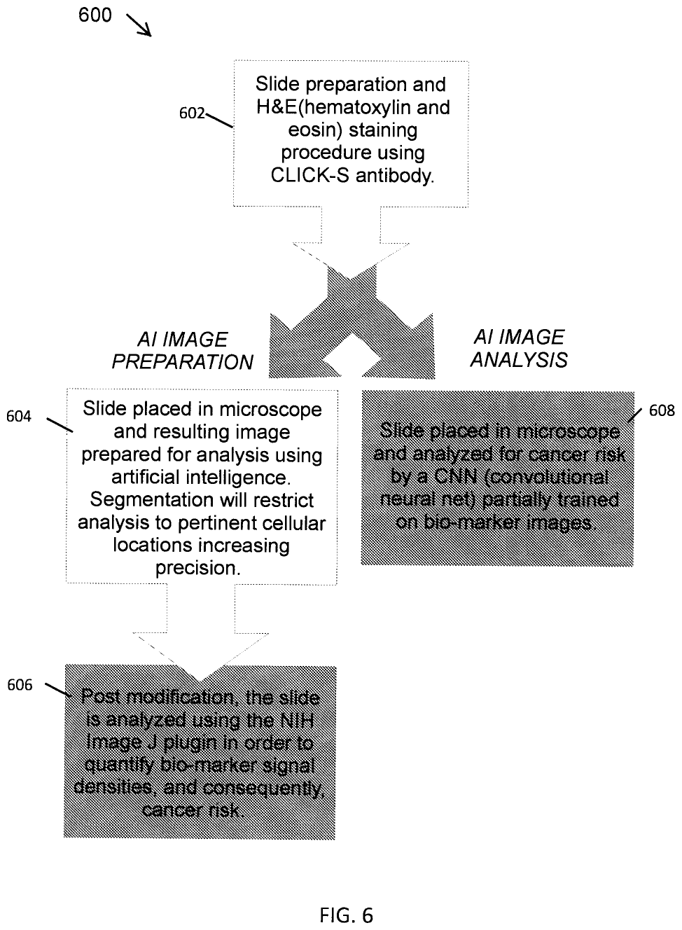

FIG. 6 is a block diagram of a method 600 in accordance with one embodiment of the invention. The slide is prepared with H&E (hematoxylin and eosin) staining procedure using CLICK-S antibody in block 602. The slide is placed in the microscope and the resulting image is prepared for analysis using artificial intelligence in block 604. Segmentation will restrict analysis to pertinent cellular locations increasing precision. In post modification, the slide is analyzed using the NIH Image J plugin in order to quantify bio-marker signal densities, and consequently cancer risk in block 606. Alternatively, the slide is placed in the microscope and analyzed for cancer risk by a CNN (convolutional neural net) partially trained on bio-marker images in block 608.

To facilitate the understanding of this invention, a number of terms are defined below. Terms defined herein have meanings as commonly understood by a person of ordinary skill in the areas relevant to the present invention. Note that these terms may be used interchangeably without limiting the scope of the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

All of the systems, devices, computer programs, compositions and/or methods disclosed and claimed herein can be made and executed without undue experimentation in light of the present disclosure. While the systems, devices, computer programs, compositions and methods of this invention have been described in terms of various embodiments, it will be apparent to those of skill in the art that variations may be applied to the systems, devices, computer programs, compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A microscope comprising:
an enclosure having an opening configured to receive a slide;
a slide holder disposed within the enclosure and operably positioned with respect to the opening to receive the slide;
a lens system disposed within the enclosure above the slide holder;
a light source disposed within the enclosure below the slide holder;
a camera disposed within the enclosure and optically aligned with the lens system;
a processor disposed within the enclosure and communicably coupled to the camera;
a display screen affixed to the enclosure and visible from an exterior of the enclosure, wherein the display screen is communicably coupled to the processor;
the processor is configured to obtain an image of a specimen disposed on the slide, analyze the specimen using artificial intelligence, and display the image of the specimen and a result of the analysis on the display screen; and
the microscope is portable.

2. The microscope of claim 1, wherein the light source comprises an addressable ring-shaped LED-based light where intensity, color and pattern are controlled by the processor for sample illumination and excitation.

3. The microscope of claim 1, further comprising:
one or more input/output connectors accessible from the exterior of the enclosure and communicably coupled to the processor; a power source disposed within the enclosure;
the display screen comprises a touch screen display; or
a memory disposed within the enclosure and communicably coupled to the processor.

4. The microscope of claim 1, further comprising an artificial intelligence processor communicably coupled to the processor.

5. The microscope of claim 1, wherein the artificial intelligence is trained to automatically prepare the image for subsequent analysis.

6. The microscope of claim 1, wherein the artificial intelligence is trained to perform an edge analysis of the specimen.

7. The microscope of claim 1, wherein the analysis comprises determining whether the image of the sample is potentially positive for a given condition and flagging the sample for further review by a subject-matter expert (SME).

8. The microscope of claim 7, wherein the potentially positive image is decreased in size.

9. The microscope of claim 7, wherein all the potentially positive images are transmitted to a device in a batch via a wired or wireless connection coupled to the processor.

10. The microscope of claim 1, wherein the processor prompts a user on how to insert the slide properly into the slide holder.

11. The microscope of claim 1, wherein the image comprises a series of images that are stitched together using the processor.

12. The microscope of claim 1, wherein the analysis comprises preselecting cellular architecture within the image by machine learning segmentation.

13. The microscope of claim 1, wherein the analysis comprises normalizing a stitched image brightness, intensity, or color of the image.

14. The microscope of claim 1, wherein the analysis comprises binning of image values to quantitate or qualify on a range of values, rather than discrete values.

15. The microscope of claim 1, wherein the artificial intelligence adjusts the image.

16. The microscope of claim 1, wherein the artificial intelligence comprises one or more qualitative or quantitative machine learning edge models.

17. The microscope of claim 1, wherein the artificial intelligence comprises a NIH Image J plugin that quantifies bio-marker signal densities and consequently cancer risk.

18. The microscope of claim 1, wherein the artificial intelligence comprises a convolutional neural net (CNN) partially trained on bio-marker images to analyze for cancer risk.

19. The microscope of claim 1, wherein the analysis comprises one or more of:
a differential White Blood Cell (WBC) count on a patient's blood smear or urine sample or bone marrow smear using Wright Stain;
a qualification of a Gram-Stained blood smear from a bacteremic patient;
a qualification of a Silver-Stained blood smear from a patient suspected of having a spirochete infection;
a qualification and quantification of a periodic acid-Schiff staining procedure on a liver sample for a patient suspected of having glycogen storage disease;
a quantification and patterning of Prussian Blue on a liver biopsy slide of a patient suspected of having hemochromatosis;
a qualification of a Gomori Trichrome Stain for a patient suspected of having liver cirrhosis;
a qualification of a Hematoxylin and Eosin(H&E) Stain on a polyp biopsy slide for a patient suspected of having cancer;
a quantification of a co-localization of multiple colors, as is the case for FRET;
a quantitation of specific cell types;
a quantification of cell morphologies; or
an assessment of tissue health.

20. A method comprising:
providing a portable microscope comprising an enclosure having an opening configured to receive a slide, a slide holder disposed within the enclosure and operably positioned with respect to the opening to receive the slide, a lens system disposed within the enclosure above the slide holder, a light source disposed within the enclosure below the slide holder, a camera disposed within the enclosure and optically aligned with the lens system, a processor disposed within the enclosure and communicably coupled to the camera, and a display screen affixed to the enclosure and visible from an exterior of the enclosure, wherein the display screen is communicably coupled to the processor;
placing the slide into the slide holder and positioning a sample on the slide within an optical path of the lens system and the camera;
capturing an image of the sample using the camera;
analyzing the sample using an artificial intelligence with the processor; and
displaying the image of the specimen and a result of the analysis on the display screen.

21. The method of claim 20, further comprising preparing the slide with a hematoxylin and eosin (H&E) staining procedure using CLICK-S antibody.

22. The method of claim 20, wherein the light source comprises an addressable ring-shaped LED-based light where intensity, color and pattern are controlled by the processor for sample illumination and excitation.

23. The method of claim 20, further comprising:
one or more input/output connectors accessible from the exterior of the enclosure and communicably coupled to the processor;
a power source disposed within the enclosure;
the display screen comprises a touch screen display; or
a memory disposed within the enclosure and communicably coupled to the processor.

24. The method of claim 20, further comprising an artificial intelligence processor communicably coupled to the processor.

25. The method of claim 20, wherein the artificial intelligence is trained to automatically prepare the image for subsequent analysis.

26. The method of claim 20, wherein the artificial intelligence is trained to perform an edge analysis of the specimen.

27. The method of claim 20, further comprising assessing the sample based on the analysis by a non-subject matter expert.

28. The method of claim 20, wherein analyzing the sample using the artificial intelligence comprises determining whether the image of the sample is potentially positive for a given condition and flagging the sample for further review by a subject-matter expert (SME).

29. The method of claim 28, further comprising decreasing the potentially positive image in size.

30. The method of claim 28, further comprising transmitting all the potentially positive images to a device in a batch via a wired or wireless connection coupled to the processor.

31. The method of claim 20, further comprising prompting a user on how to insert the slide properly into the slide holder.

32. The method of claim 20, further comprising stitching together a series of images together into the image using the processor.

33. The method of claim 20, wherein analyzing the sample using the artificial intelligence comprises preselecting cellular architecture within the image by machine learning segmentation.

34. The method of claim 20, wherein analyzing the sample using the artificial intelligence comprises normalizing a stitched image brightness, intensity, or color of the image.

35. The method of claim 20, wherein analyzing the sample using the artificial intelligence comprises binning of image values to quantitate or qualify on a range of values, rather than discrete values.

36. The method of claim 20, wherein analyzing the sample using the artificial intelligence comprises adjusting the image.

37. The method of claim 20, wherein the artificial intelligence comprises one or more qualitative or quantitative machine learning edge models.

38. The method of claim 20, wherein the artificial intelligence comprises a NIH Image J plugin that quantifies bio-marker signal densities and consequently cancer risk.

39. The method of claim 20, wherein the artificial intelligence comprises a convolutional neural net (CNN) partially trained on bio-marker images to analyze for cancer risk.

40. The method of claim 20, wherein analyzing the sample using the artificial intelligence comprises one or more of:
performing a differential White Blood Cell (WBC) count on a patient's blood smear or urine sample or bone marrow smear using Wright Stain using the artificial intelligence;
performing a qualification of a Gram-Stained blood smear from a bacteremic patient using the artificial intelligence;
performing a qualification of a Silver-Stained blood smear from a patient suspected of having a spirochete infection using the artificial intelligence;
performing a qualification and quantification of a periodic acid-Schiff staining procedure on a liver sample for a patient suspected of having glycogen storage disease using the artificial intelligence;
performing a quantification and patterning of Prussian Blue on a liver biopsy slide of a patient suspected of having hemochromatosis using the artificial intelligence;
performing a qualification of a Gomori Trichrome Stain for a patient suspected of having liver cirrhosis using the artificial intelligence;
performing a qualification of a Hematoxylin and Eosin(H&E) Stain on a polyp biopsy slide for a patient suspected of having cancer using the artificial intelligence;
performing a quantification of a co-localization of multiple colors, as is the case for FRET using the artificial intelligence;
performing a quantitation of specific cell types using the artificial intelligence;
performing a quantification of cell morphologies using the artificial intelligence; or
performing an assessment of tissue health using the artificial intelligence.
